(19) United States
(12) Patent Application Publication
(10) Pub. No.: US 2012/0301869 A1
(43) Pub. Date: Nov. 29, 2012

(54) PARTICLE SEPARATION DEVICES, METHODS AND SYSTEMS

(75) Inventor: Kenneth Michael Evans, College Station, TX (US)

(73) Assignee: INGURAN, LLC, Navasota, TX (US)

(21) Appl. No.: 13/481,622

(22) Filed: May 25, 2012

Related U.S. Application Data
(60) Provisional application No. 61/489,996, filed on May 25, 2011.
(60) Provisional application No. 61/506,918, filed on Jul. 12, 2011.

(51) Int. Cl. C12M 1/42; C12M 1/00; C12N 5/076

(52) U.S. Cl. 435/2; 435/288.7; 435/283.1

(57) ABSTRACT

A device, system and method for the separation and collection of sperm cells into multiple subpopulations based upon sperm characteristics and for facilitating the collection of multiple subpopulations in limited space. The device can be a redirection device including one or more spaced apart tubes, where each tube has a tube inlet for collecting particles in a flow path, a tube outlet for dispensing the collected particles, and a tube body connecting the tube inlet to the tube outlet for redirecting the particles in the flow path. The device can further include a support for holding each tube in a spaced apart relationship and a securing element for securing the one or more spaced apart tubes to the support.

The present non-provisional patent application claims priority under 35 U.S.C. §119(e) to co-pending U.S. Provisional Application Ser. No. 61/489,996and co-pending U.S. Provisional Application Ser. No. 61/506,918, the full disclosures of both are incorporated herein by reference.

### TECHNICAL FIELD

The present invention relates generally to particle separation devices, methods and systems and more particularly to the separation and collection of sperm cells into multiple subpopulations based upon sperm characteristics and to facilitating the collection of multiple subpopulations in limited space.

### BACKGROUND

Methods and devices exist in the field of flow cytometry for the separation and collection of particles, such as cell sorting. In particular, jet-in-air flow cytometers have been used for sex-sorting X- and Y-chromosome bearing subpopulations of sperm as described in U.S. Pat. Nos. 5,135,759, 7,371,517, and 7,758,811, each of which is incorporated herein by reference.

One such jet-in-air flow cytometer adapted for sorting sperm, is available as the MoFlo™ XDPSX, from Beckman Coulter (Fort Collins, US). This modified instrument is able to differentiate sperm characteristics based upon differences in DNA content. In particular, sperm cells which are stoichiometrically stained with a DNA selective dye differentially fluoresce in response to an excitation energy source based upon the typical mammalian 2-4% difference in DNA content between X- and Y-chromosome bearing sperm. Such a flow cytometer is able to identify the different characteristics of the sperm cells contained in a fluid stream based upon the difference in fluorescence and is able to provide droplets formed from the fluid stream with a predetermined charge.

Droplets containing X-chromosome bearing sperm cells could be given a first charge and drops containing Y-chromosome bearing sperm cells could be given a second charge. The stream of individually charged droplets then passes between a pair of electrostatically charged plates causing individual droplets to be deflected into divergent flow paths determined by their charge.

Several significant problems exist with the application of this technology to sperm which results in a large percentage of viable sperm being discarded as waste.

One significant problem exists in that the relatively small differences in sperm DNA content are difficult to distinguish, leaving a significant portion of a sperm sample unidentified as either X- or Y-chromosome bearing sperm. In order to differentiate, in bovine for example, a 3.8% difference in DNA content, each sperm cell in the sample must be uniformly stained which a DNA binding fluorochrome dye and precisely orientated at the detector. Thus, slight variations in stain uniformity and sperm cell orientation introduce variation into two closely related subpopulations. These two factors combined with the fact the DNA difference is already very small between the two populations causes a degree of overlap between the subpopulations of sperm cells. In instances where the desirable purity is greater than 95%, fewer sperm can be determined with the requisite confidence level as compared to 70% 80% or 90% purities, meaning fewer sperm are sorted at increasingly high purities and that more viable sperm cells are disposed with the waste stream.

Another significant problem exists with respect to discarding viable sperm cells due to the occurrence of coincident events. A coincident event occurs when two or more sperm cells are too close together within the fluid stream for the identification of their DNA characteristics, or when two or more sperm are too close together to be reliably separated. In either event, all of the sperm cells may be discarded with waste, whereas some or all of those discarded cells may have been desirable to collect.

In some fields these problems are overlooked in view of raw throughput. For example, in the case of bovine sperm, it is relatively easy to collect and process, and high purities can be desirable in both the beef and dairy industries, even at the expense of discarding the majority of the sperm sample.

However, this high throughput methodology is not acceptable for sperm in limited supply. For example, a specific animal such as bovine, equine, cervine, porcine or other livestock could possess exceptionally desirable genetic qualities, but may produce poor sperm samples for sorting. A species could be rare, endangered, or difficult to collect, limiting the amount of sperm available for sorting. A previously collected sample may be preserved, but the animal or species may no longer be available for subsequent collections. Regardless of the circumstances, the wasteful sperm sorting process is undesirable for sperm in limited supply or sperm with high value.

### SUMMARY OF THE INVENTION

In view of the deficiencies that exist in the prior methods and devices, a need exists for a device, method and system to improve sorting efficiency, particularly for particles which are expensive or in limited supply. Accordingly, a broad object of the present invention can be to provide improvements in sorting sperm cells which meet the needs set forth above. The improvements may include a device, method and system for sorting sperm in three streams and collecting three subpopulations of sperm including a subpopulation enriched for X-chromosome bearing sperm, a subpopulation enriched for Y-chromosome bearing sperm, and a subpopulation containing the remaining live sperm.

In one embodiment, the present invention relates to an apparatus having two or more spaced apart tubes for redirecting streams of droplets. Each tube can have a tube inlet for receiving a stream of droplets in a flow path connected, with a tube body, to a tube outlet for dispensing the collected droplets. A securing element may secure each tube to a support, which may hold each tube in a spaced apart relationship.

In one embodiment involving three tubes, the apparatus enables a conventional flow cytometer to run a three stream sort of sperm cells into three standard collection vessels. In the three stream sort, the apparatus enables X-chromosome bearing sperm to be redirected from a first trajectory to a first collection vessel, Y-chromosome bearing sperm to be redirected from a second trajectory to a second collection vessel, and substantially the remaining live sperm cell population to be redirected from a third trajectory to a third collection vessel, while waste, including dead or dying sperm cells, remains undeflected.

Another embodiment relates to a flow cytometer system for isolating three or more subpopulations of cells. The flow cytometer can include a nozzle for producing a fluid stream along a flow axis as well as an oscillator for breaking the fluid stream into droplets. A laser may be provided to interrogate cells within the fluid stream and a detector can detect emitted or reflected electromagnetic radiation from each cell in response to laser interrogation. An analyzer may determine cell properties from the emitted or reflected electromagnetic radiation. The analyzer can identify cells as: alive and having a first characteristic, alive and having a second characteristic, alive and undetermined with respect to the first and second characteristics, or dead. A charge circuit may charge droplets as they form based on the identification provided by the analyzer such that droplets containing live cells with the first characteristic are provided with a first trajectory, droplets containing live cells with the second characteristic are provided with a second trajectory, and droplets containing live cells with neither the first nor the second characteristic are provided with a third trajectory. A particle redirection device may be aligned to receive droplets in each of the first trajectory, second trajectory, and third trajectory. A first tube of the particle redirection device may be aligned to receive droplets in the first trajectory and dispense them in the first collection area. A second tube of the particle redirection device may be aligned to receive droplets in the second trajectory and dispense them to a second collection area. A third tube of the particle redirection device may be aligned to receive droplets in the third trajectory and dispense them to a third collection area. A support may hold each tube in a spaced apart relationship.

Still another embodiment of the invention relates to a method of sorting and collecting three subpopulations of sperm cells. The method may begin with the step of producing a fluid stream containing living sperm cells with a first characteristic, living sperm cells with a second characteristic, and dead sperm cells. The method may continue with detecting properties of sperm cells in the fluid stream. A first subpopulation of sperm having a first characteristic, a second subpopulation of sperm having a second characteristic, dead or dying sperm, and a third subpopulation of sperm which are neither dead nor identified as having the first characteristic or the second characteristic may each be identified. The first, second and third subpopulations may be individually collected, while the dead sperm is discarded with waste.

### BRIEF DESCRIPTION OF THE DRAWINGS

FIG. 1 illustrates one embodiment of a particle redirection device according to the present invention.

FIG. 2 illustrates one embodiment of a particle redirection device according to the present invention.

FIG. 3 illustrates another view of the particle redirection device depicted in FIG. 2.

FIG. 4 illustrates the particle redirection device depicted in FIGS. 1 and 2 fitted to an X-Y-Z stage allowing the particle redirection device to be adjusted.

FIG. 5 illustrates a flow cytometer system of the present invention generating four streams, one uncharged waste stream, and three streams of charged particles, each charged stream being directed into a tube inlet of the particle redirection device of FIG. 4 connected to an X-Y-Z stage, each stream subsequently being collected in a collection container held in housings, both the containers and housings also forming part of the system of the present invention.

FIG. 6 illustrates a schematic representation of part of the flow cytometer system depicted in FIG. 5 illustrating a method of generating three charged fluid streams and directing the streams into respective tube inlets of a particle redirection device according to the present invention.

### MODES FOR CARRYING OUT THE INVENTION

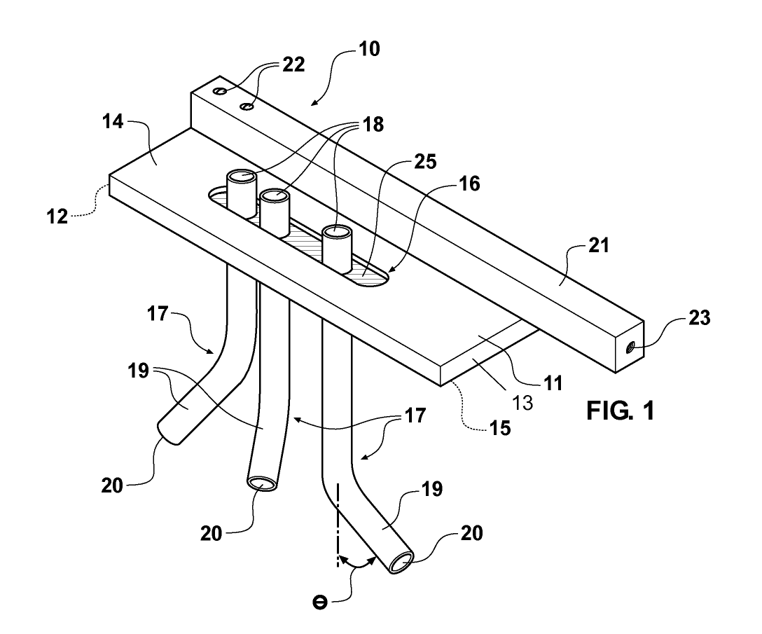

Turning now to FIG. 1 a particle redirection device 10 is illustrated for use with a sorter, such as a jet-in-air flow cytometer. The device 10 can have a support 11 which may be constructed from an elongate rectangular metallic structure or another suitably rigid and sturdy material. The support 11 can have parallel front 12 and rear 13 faces and parallel upper 14 and lower 15 faces. However, other geometries are also contemplated herein so long as the support 11 provides a sufficient area on the upper face 14 to retain two or more spaced apart tubes, and so long as the overall structure of the support 11 is sturdy enough to maintain those tubes in a rigid spaced apart relationship. An elongate slot 16 can be located within the support 11 extending through the support upper face 14 and lower face 15 and running parallel to the support longitudinal axis.

Three tubes 17 can be located within the slot 16. In one embodiment, each tube 17 can be constructed as a spaced apart elongate stainless steel tube with a circular cross-section and can be maintained in position by a securing element. In FIG. 1 the securing element is depicted as a resin 25, such as an electrically conductive which fills the slot 16 securing the tubes 17 with the support 11. The resin 25 may be a cross-linked electrically conductive resin. FIG. 1 illustrates two of the tubes 17 can be located in a closer proximity towards one end of the resin 25 filled slot 16 while the remaining tube 17 is located towards the other end of the resin 25 filled slot 16. However, the tubes 17 may be spaced apart equally, or otherwise secured in any number spatial arrangements.

Each of the tubes has a tube inlet 18 and a tube outlet 20 connected through a tube body 19, which may be a bent tube body. Each of the tube inlets 18 can be elevated relative to the upper face 14 of the support 11 and can be substantially flush with respect to each other. Having the tube inlets 18 elevated relative to the upper face 14 of the support 11 may help prevent cross contamination of charged droplets which may have landed on the support upper face 14 from subsequently entering any of the tube inlets 18.

Below the lower face 15 of the support 11, each tube body 19 may have a bend having a bend angle. In one embodiment, the bend of each tube is similar, while in another embodiment the bend of all the tubes is not similar. FIG. 1 illustrates a bend angle 0 of about 55 degrees with respect to the vertical. The portion of the inner tube 17 immediately above the upper face 14 and immediately below the lower face 15 of the support 11 extends at right angles to the support 11.

FIG. 1 depicts the tube outlets 20 as not parallel with respect to each other. However, in an alternative embodiment, the tubes could diverge and subsequently bend back into a parallel configuration.

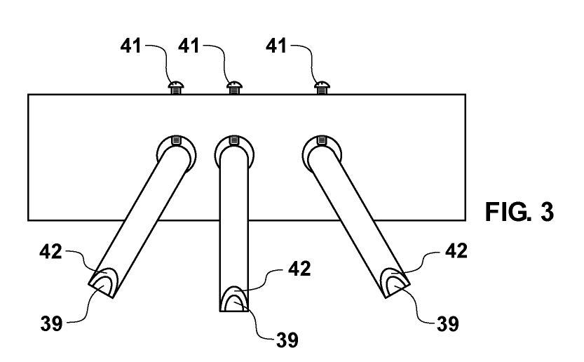

An elongate arm 21 can be fixed to the upper face 14 of the support 11 beyond the rear of the slot 17 by fasteners 22. The fasteners 22 can include, bolts, screws, nails, pins, adhesives and other similar means. An aperture 23, such as a threaded aperture, may be located in one end of the arm and parallel with its longitudinal axis enables the support 11 to be fitted to an adjustable X-Y-Z stage 44 (See FIG. 3) via a complementary screw thread located thereon enabling coarse adjustment of the position of the device 10 to made, when in use.

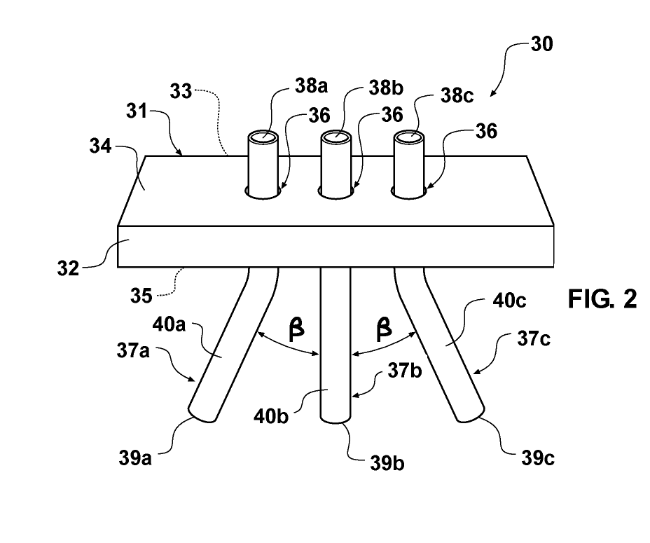

FIG. 2 illustrates an alternative embodiment of the particle redirection device 30. Some features are common between the embodiments of FIG. 1 and FIG. 2, and for these features, the description of FIG. 1 can be understood to apply to FIG. 2 and the description of FIG. 2 can be understood to apply to FIG. 1, where appropriate. A support 31 is depicted having parallel front 32 and rear 33 faces and parallel upper 34 and 35 lower faces, however, other shapes are also contemplated as long as enough space is provided on the upper face 34 to retain each desired spaced apart tube 37. The support 31 may be constructed from an extruded metal or another suitably rigid and sturdy material. Extending vertically through the upper 34 and lower 35 faces and bisecting the longitudinal axis of the support 31 are three similar spaced apart apertures 36. Two or more spaced apart tubes 37 may be placed within the apertures 36 in a sliding arrangement. The spacing of the apertures 36 defines the spacing of the two or more spaced apart tubes 37. As but one example, a first tube 37a, a second tube 37b and a third tube 37c may be spaced apart in respective apertures 36. The two or more spaced apart tubes 37a, 37b, 37c can each be elongate stainless steel circular tubes having tube inlets 38a, 38b, 38c and tube outlets 39a, 39b, 39c connected by a bent tube body 40a, 40b, 40c. Naturally, the tube may be constructed with different cross-sectional shapes and from different suitable materials. In one embodiment, the tubes 37 are constructed from an electrically conductive material. Each tube 37 can be maintained within the aperture 36 by a securing element illustrated as screw 41 (seen in FIG. 3). A threaded sleeve may extend from the rear face 33 of the support 31 to the wall of the aperture 36, through which the screw 41 may be adjusted into contact with the tube 37. In one embodiment, each respective threaded sleeve is concentric with the center of the aperture 36. The shape of the apertures 36 can generally match the cross section of the tubes 37, and can be, for example, circular, but may also be polygonal or elliptical.

Accordingly, unlike in the first embodiment, the tubes 37 illustrated in FIG. 2 can be easily removed, rotated within their respective aperture 36 and/or have their vertical position adjusted by loosening and tightening the screw 41 in the threaded sleeve.

Below the lower face 35 of the support 31 each tube body 39 can have a similar bend with a bend angle β from the vertical, which as an example can be substantially 60 degrees. As with the first embodiment, the distance separating the inner from the two outer tubes 37 is different making two of the tubes 37 closer together with respect to the other.

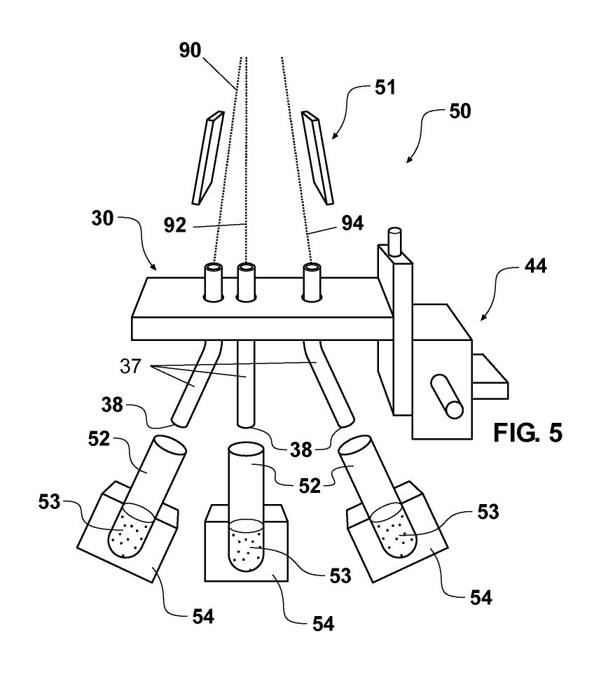

In other embodiments, one or more tubes may be wider in diameter than the others and such an arrangement may be particularly advantageous for the tube which is set apart from the other two. When used to sort sperm, the two tubes of the device which are closest together can receive a positively charged X-chromosome bearing stream and a less positively charged Y-chromosome bearing stream whilst the remaining tube is can receive the remaining stream containing the live sperm cell population that could not be identified as either of the previous two categories to be collected (FIG. 1 and FIG. 5). This last stream can have a wider diameter than the previous two streams as it will contain a higher frequency of droplets than the other two streams.

Due to the wider diameter of the stream and the higher frequency of the drops contained within it, the chances of an errant droplet emanating from this stream is greater than the other two streams. Accordingly, setting this tube apart from the others reduces the possibility of such an errant drop entering one of the other tubes and cross contaminating one of the other streams.

In an embodiment illustrated in FIG. 3, each tube outlet 39 contains a substantially “V” shaped groove 42 (although in alternative embodiments the groove could be substantially “U” or “C” shaped) located on its underside making them larger than the tube outlets 20 of the first embodiment. Such embodiments may enable droplets forming in the tube outlet 39 to be larger than a corresponding tube outlet 20 (illustrated in FIG. 1) and breaking the surface tension of such a droplet by tapping it with the mouth of a collection vessel may drag the entire drop into the collection vessel allowing the collection vessel (if now full) to be replaced with an empty collection vessel before another droplet has had a chance to fully form at the tube outlet 39. Such an arrangement means that the particle collection may not need to be stopped in order to change collection vessels.

In an alternative embodiment, the tube outlet could possess a taper which could either widen or narrow the size of the tube outlet depending on the type of taper. A widening taper may also provide the same effect as groove 42 described above.

The embodiment of FIG. 2 omits the elongate arm of FIG. 1, and instead includes a threaded aperture 43 in one end of the support 31. This threaded aperture 43 allows the support 31 to be directly fitted to an X-Y-Z stage 44 via a complementary screw thread located thereon.

In additional embodiments, a particle redirection device may be configured to redirect four or more streams of droplets.

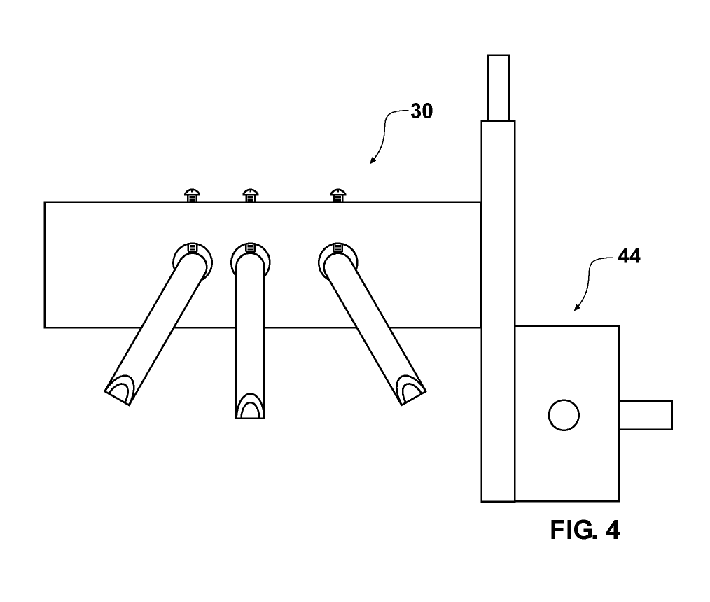

FIG. 4 illustrates the device 30 fitted to an X-Y-Z stage 44 enabling the coarse adjustment of the device 30 when it is used in conjunction with a flow cytometer or cell sorter to redirect particles. In one embodiment, a video camera (not illustrated) could be used to enable an operator to monitor particle sorting more clearly on a video screen and to better see what types of coarse adjustments need to be made to the device 30 using the X-Y-Z stage 44. Additionally, the video screen may enable an operator to see what types of fine adjustments need to be made to the potential difference across the deflector plates to move and control the streams to ensure that each droplet stream is entering respective tube inlets in the most desirable way. The video camera could be mounted or be mountable to the support, the cell sorter or to the flow cytometer.

In alternative embodiments, the support could be provided with means to enable it to be self supporting without the need for an X-Y-Z stage 44. For example, the support 31 could be provided with a pair of integral or detachable legs. Such an embodiment would probably not be able to be adjusted as accurately as one connected to an X-Y-Z stage 44, however, if the size of the tube inlets were tapered outwards to widen the mouth of the inlet or if wider tubes were used, then such a fine adjustment may not be required.

Another embodiment of the device is envisioned which incorporates features of FIG. 1 and FIG. 2, where the slot 16 from the first embodiment is used but the tubes are secured within the slot using the screws 41 of the second embodiment. In such an arrangement, the support would contain a plurality of parallel threaded sleeves extending from the rear face of the support to the wall defining the slot. A tube could then not only be height or rotation adjusted (or even removed) when a screw 41 is loosened and tightened but as a screw could be placed within any one of the complementary sleeves along the length of the support, the position of one or more of the tubes along the length of the slot may also be adjusted.

In yet a further embodiment, tubes from several devices of the present invention may be directed into a single collection tube. This may be accomplished for example, by setting up three flow cytometers in a row and using three of the devices set up in the manner previously described, one below the deflection plates of each of the flow cytometers. However, instead of the tubes in each device being approximately the same length, one of the tubes on either side of the central flow cytometer can be replaced with one long enough that its tube outlet overlies the mouth of a single 50 ml collection tube located under the central flow cytometer. In this embodiment, the tube outlets of three tubes can overlie the mouth of a single collection vessel and the arrangement between the flow cytometers and these three tubes could be such that each of these tubes redirects the same type of particle from each flow cytometer e.g. droplets containing an X-chromosome bearing sperm cell from a common supply e.g. ejaculate from the same mammal. Of course the ejaculate used in this embodiment may also be from three different mammals from the same species, each flow cytometer sorting the sperm from each of the different mammals. Instead of using three devices of the types illustrated herein to achieve collection of one type of particle such as an X-chromosome bearing sperm, a single device if designed correctly spanning three flow cytometers in a row could be used. Clearly there will be a limit to the number of flow cytometers that could be used to supply a single collection vessel as the surface area of the collection vessel mouth will only be able to accommodate a certain number of tube outlets.

However, irrespective of whether a single device or multiple devices associated with multiple flow cytometers are used, the principle of multiple flow cytometers supplying a single collection vessel with the same type of particle has been established. In this way the device of the present invention may be used as part of a system to isolate three or more populations of particle being supplied from either a single flow cytometer or three or more populations (the actual number will depend on the constraints of the surface area of the mouth of a collection vessel and the surface area of the tube outlets) from multiple flow cytometers.

The bend in each tube may serve two functions, the first function may be to redirect a particle stream that enters the body of the tube via the tube inlet and the second is to provide a relatively cushioned impact zone for such particles when they impact the bend to minimize damage to the particles. For this reason the bend angle θ is a relatively shallow which could be in the range of 40 degrees to 80 degrees with respect to the vertical. It could also be in the range of 50 to 70 degrees with respect to the vertical and may also be in the range of 55 to 60 degrees with respect to the vertical.

Although in actuality, some particles may initially impact the inner wall of a tube prior to impacting the bend, the main impact zone for particles is intended to be the bend. As a particle stream impacts the impact zone, there will be a reduction in the velocity of the stream. This reduction in velocity will cause a slight “pooling” or “build up” of particles within the impact zone. Once formed, the pooling will be in a state of flux but may provide a fluid “cushion” for subsequent particles as they impact the zone.

In another embodiment, the impact zone may constitute a “U” bend in the tube or may be a “bulb” or “well”, which will fill with particles and once filled, the overflow of particles will continue along within the tube body towards the tube outlet. In either embodiment, as particles will be impacting a fluid impact zone, damage to the particles is likely to be reduced compared with hitting a solid wall. Furthermore (and as mentioned hereinabove) if the tube outlet were to have a taper and if it were a narrowing taper, the size of the tube outlet will be reduced in size and this size reduction may enable a greater back pressure of fluid to be set up within the tube body which will increase the depth of the pooling in the impact zone of those embodiments only having a bend of the type illustrated in FIG. 1 and FIG. 2. In other non illustrated embodiments, the tubes may have a second bend going in the opposite direction to the first (forming an “S” type shape) and located towards the tube outlet so that that portion of the tube towards the tube outlet may appear similar to a faucet depending on the degree of bend. The angle of this second bend may reduce the distance of travel that a drop leaving the tube outlet has to travel before it contacts the inner side wall of a collection vessel reducing any damage to the particle which may be caused by the velocity of such an impact.

The tubes can be made of an electrically conductive material as they are designed to redirect electrically charged particles and thus need to be electrically grounded when in use. In this regard although stainless steel could be used, titanium and a suitable reproductive biocompatible material could also be used.

Similarly, although a metallic electrically conductive support has been described, the support 31 need not be metallic and other materials which are electrically conductive such as materials considered to be non-metallic electrical conductors such as graphite may be used instead. Alternatively, a plastics support could be employed if it were coated with an electrically conductive polymer or covered in electrically conductive paint or a metallic sheet to enable the tubes to be electrically grounded via the support. Having a support made of an electrically conductive material can provide an advantage as it is easier to electrically ground the support rather than having the support made of non-electrically conductive plastics material and having to ground each tube individually.

Another aspect of the present invention relates to a system of isolating three or more populations of particles using a cell sorting instrument and of those available to form part of such a system, one embodiment using a flow cytometer is depicted in FIG. 5.

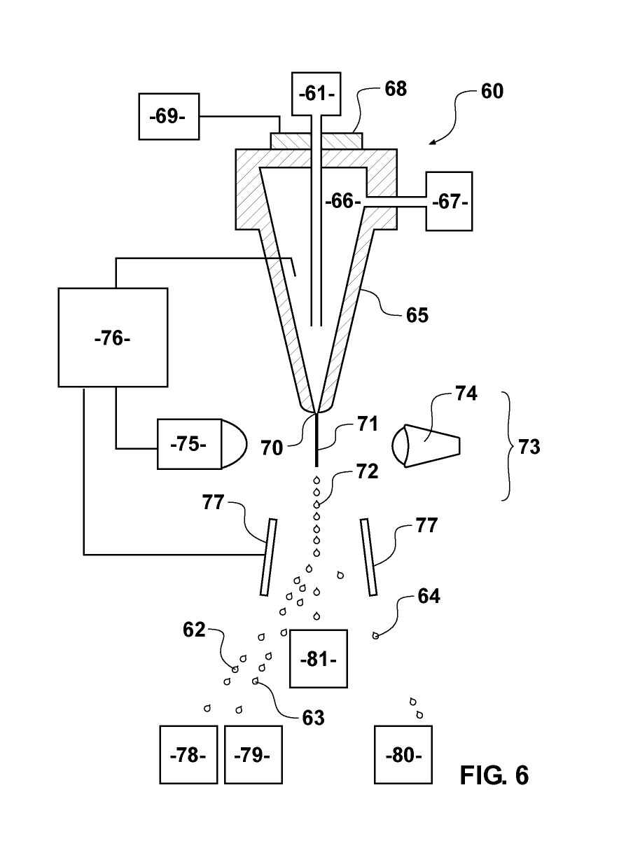

FIG. 5 illustrates a portion of a flow cytometer system 50 being used to collect three streams of droplets containing particles, such as sperm cells. A first stream of droplets 90 is illustrated with a first trajectory, a second stream of droplets 92 is illustrated with a second trajectory and a third stream of droplets 94 is illustrated with a third trajectory. The illustrated portion of the system 50 includes deflection pates 51, which forms the three streams of droplets by creating an electrical field to which the individually charged droplets are responsive. Individual droplets are deflected by the plates 51 to follow the trajectory of one of the three streams of droplets based upon a charge applied to each individual droplet. An undeflected waste stream is not depicted in this figure, but can be seen in FIG. 6. A particle redirection device 30 may be affixed to the adjustable X-Y-Z stage 44 for positioning to capture each of the three streams of droplets. Each stream is captured by a different one of the three spaced apart tubes 37. A first tube redirects droplets in the first trajectory to deposit droplets at a first collection location, at which a first vessel is illustrated for collecting those particles. The second tube redirects droplets in the second trajectory to deposit droplets stream to a second collection location into a second collection vessel. Similarly, a third tube redirects the third stream of droplets to a third location and into a third collection vessel. Each of the collection vessels may be 50 ml collection vessel 52 and may contain catch media 53, and three separate housings 54, each housing containing one of the collection vessels 52, the longitudinal axis of the housing being substantially parallel with the longitudinal axis of the collection vessel when the housing contains a collection vessel. In this way three streams may be sorted, whereas three conventional 50 ml collection vessels do not fit in the collection area of standard flow sorters (MoFlo SX and MoFlo XDPSX).

Each of the housings 54 may be adapted to be able to rest in one of two orientations, either on its base or (as illustrated) at angle relative to its base. In this second (non-base) orientation, each tube outlet 38 is able to sit within the mouth of a respective 50 ml collection vessel 52. The system illustrated could also comprise the video camera previously described for monitoring particle sorting when in use either mounted or mountable to the device or the flow cytometer. Of course, the system may simply comprise, as an absolute minimum, a cell sorter or a flow cytometer together with the device.

If a four way sort were required, the device would need four tubes and four collection vessels and four housings.

In an embodiment, the system may include a single housing adapted to house all of the collection vessels. Such a housing may be able to rest in one of two orientations or it may only rest in one intended orientation, the recesses for the collection vessels being set at an angle relative to the base of the housing. Having a housing able to rest on its base and at an angle with respect to its base could enable the housing to be used as part of the system of the present invention when not resting on its base, and on its base when being used for normal particle sorting or collection. The second orientation may be at 45 degrees to the base. In fact it may lie in the range of 30 to 60 degrees relative to the base or it may lie in the range of 40 to 50 degrees relative to the base.

Another aspect of the present invention includes a method of using flow cytometer system to isolate three or more populations of particles and one embodiment of the method is depicted by FIG. 6.

FIG. 6 illustrates in schematic form part of the flow cytometer system shown in FIG. 5, used to sort sperm cells and the tube inlets of the device of the present invention and is generally referenced 60.

The flow cytometer 60 illustrated in FIG. 6 includes a sperm cell source 61 which supplies a sample containing sperm cells stained with both a fluorochrome dye and a quenching dye for analysis and/or sorting by the flow cytometer 60. Initially, the sample is deposited into the nozzle 65 under pressure and is coaxially surrounded by a sheath fluid 66 supplied to the nozzle 65 by a sheath fluid source 67 for producing a fluid stream 71 from the nozzle 65. An oscillator 68 which may be precisely controlled with an oscillator control mechanism 69 to create pressure waves within the nozzle 65 which are transmitted to a fluid stream 71 as it leaves the nozzle orifice 70. As a result, the fluid stream 71 may be produced along a flow axis in the form of a coaxial stream eventually and regularly forming droplets 72 of sample and sheath fluid.

The charging of the respective droplet streams is made possible by the cell sensing system 73 which includes a laser 74 that illuminates or interrogates the fluid stream 71. Cells within the fluid stream 71 emit or reflect electromagnetic radiation in response to the laser 74, and this emitted or reflected electromagnetic radiation can be detected by a detector 75. The information received by the detector 75 is provided to an analyzer 76 which very rapidly makes the decision as to whether to charge a forming droplet, and if so, which charge to provide the forming drop and then charges the droplet 72 accordingly.

The charged or uncharged droplet streams then pass between a pair of electrostatically charged plates 77, which cause them to be deflected either with a particular trajectory depending on their charge. Three different trajectories are illustrated for depositing droplets into electrically grounded tube inlets 78, 79 and 80 of a device redirection device. The uncharged non-deflected sub-population stream containing empty droplets and droplets with dead cells (or those about to die) go to the waste container 81.

With specific reference to sperm cells, a characteristic of X-chromosome bearing sperm is that they tend to absorb more fluorochrome dye than Y-chromosome bearing sperm and as such, the amount of light emitted by the laser excited absorbed dye in the X-chromosome bearing sperm differs from that of the Y-chromosome bearing sperm. This difference, as detected by the detector 75, provides the analyzer 76 with enough information to identify sperm cells and to coordinate a charge circuit to charge the respective droplets in which the sperm cells are contained. These droplets are readily distinguishable from those containing mixtures of live sperm cells which are charged differently. Dead cells (or those about to die) have absorbed the quenching dye and the analyzer 76 does not charge droplets containing such cells. As previously described, those droplets containing cells which cannot be identified with the required confidence level may be provided with a different charge.

The flow cytometer 60 can be programmed by an operator to generate three charged droplet streams having three different trajectories. As one example, the first droplet stream 62 containing droplets having X-chromosome bearing sperm cells can be charged positively to some specified value. This charge causes each droplet determined to have an X-chromosome bearing sperm cell to be deflected into a droplet stream with a uniform first trajectory. A second droplet stream 63 containing Y-chromosome bearing sperm cells can be formed by providing a uniform but differing charge to those droplets containing Y chromosomes bearing sperm. As one example, the second stream may be charged to a lower positive value as compared to the specified value of the first droplet stream, resulting in reduced angle of trajectory in the same direction as the first droplet stream. Dead or dying sperm, which can be determined by the uptake of certain dyes, may remain neutral for a trajectory which remains substantially aligned with the initial flow axis for collection with the waste, or empty droplets.

Finally, a third droplet stream 64 containing a mixture of X- and Y-chromosome bearing sperm cells 64 (excluding dead, or transitioning—i.e. those sperm cells about to die) can be charged negatively to provide a third trajectory. The third droplet stream 64 may contain those live X- and Y-chromosome bearing sperm which could not be identified with the requisite confidence for sorting in the X- and Y-chromosome bearing sperm subpopulations. The percentage droplets in the third stream of droplets may depend on the desired purity of the X and/or Y chromosome bearing subpopulations.

Taking the X-chromosome bearing droplet stream 62 as an example, the droplet stream once it has entered the tube inlet 78 continues to travel within the body of the tube and upon impacting the bend within an impact zone of the tube body the droplet stream's velocity is reduced before being redirected within the tube body. The reduction in velocity of the droplets upon impacting the bend of the tube may cause a slight “pooling” or “build up” of fluid from the droplets (previously described herein) within the impact zone. The pooling once formed, is in a state of flux but nevertheless, provides a fluid “cushion” for subsequent droplets as they impact the zone.

Although such cushioning tends to reduce any damage to the sperm in the droplet stream as the droplets impact the zone, the initial sperm containing droplets would not be provided with that luxury so it is possible to provide a slight initial pooling by programming the flow cytometer to initially only allow charged sheath fluid (empty i.e. non-sperm containing droplets of sheath fluid) to enter the tube inlets before subsequently allowing sperm cells to be coaxially surrounded by sheath fluid and subsequently sorted in droplets.

The X-chromosome bearing droplet stream 62 within the tube body continues to travel towards the tube outlet and eventually due to surface tension, forms larger drops which drip from the tube outlet of the tube at a first collection location into a sperm collection vessel, such as a 50 ml collection vessel. The collection vessel contains catch media and is located within a housing which has been rested in its non-base orientation. The same events occur for the other charged droplet streams.

Once a collection vessel is judged to be full enough, an operator can pick up the collection vessel and allow the mouth edge of the collection vessel to touch a drop forming at the mouth of the tube outlet which has the effect of breaking the surface tension of the drop and “dragging” the surface tension broken drop into the collection vessel.

This procedure allows the collection vessel to be replaced with an empty vessel (save for the catch media contained therein) located within a similar housing before another drop has had an opportunity to form and subsequently exit the tube outlet of the tube.

Accordingly, with such a procedure in combination with the use of the particle redirection device, the sorting process does not need to be halted to enable a filled collection tube to be replaced. Each type of the collected sorted sperm is then dealt with using established protocols.

As the method allows all of the live sperm to be collected, the procedure tends to minimize waste and the un-sex sorted collected sperm cells which may be financially valuable because of limited supply or genetic pedigree (such as the White-tailed deer, Moose or Eld's deer) may then be sold for the purposes of research or for artificial insemination to those individuals or breeders who do not mind what the sex of the offspring is.

### EXAMPLE

### Semen Collection

Semen from White-tailed deer bucks was collected via electroejaculation and portions of the ejaculate were collected, kept separate, and analyzed based on consistency and color. The portions with the best motility (>˜80%), based on subjective microsope measurements, were extended to approximately 1×10⁹ sperm/ml with Biladyl® or Tris A solution (200 mM Tris, 65.7 mM citric acid, 55.5 mM fructose) containing 20% (v/v) egg yolk and adding 7% (v/v) glycerol for cryopreservation. All the samples were shipped or driven to Sexing Technologies, Navasota, Tex., USA for processing during the first 24 hours after collection. Samples were chilled and maintained between 5-10° C. during transport.

For semen selection the standards for routine semen preparation and cut-off values for standard semen characteristics for selecting the ejaculates for processing were applied, however deer semen was processed regardless of concentration, determined using the SP1-Cassette, Reagent S100, and NucleoCounter® SP-100™ system.

Each conventional sorted and sex-sorted sample obtained through three way sorting (with the dead going to waste) was produced according to the standards for semen production at Sexing Technologies. Three way sorting of X- or Y-chromosome bearing sperm and unsorted live sperm (the latter being collected as the third stream) was conducted using MoFlo™ XDPSX sperm sorters and Summit v5.0 software (available from Beckman Coulter, Fort Collins US). For these experiments, the MoFloυ XDPSX was set to a sorting pressure of 40 psi.

More specifically, neat semen samples were diluted to 160×10⁶/mL in modified Tyrode's albumin lactate pyruvate (TALP) stained with 16 gl/ml of Hoechst 33342 fluorochrome dye diluted 1:1 (v/v) in milli-Q water (8.1 mM) and incubated for 45 minutes, and then red TALP was added (TALP+4% egg yolk+0.002% FD&C #40 red food dye) to get a concentration of 80 M/ml, afterwards samples were filtered using a Partec CellTrics® 50 μm disposable filter and placed in 5 ml polypropylene tubes for sorting.

The laser source was a NdYag mode locked pulsed Vanguard™ operating at 355 nm. The operating power was light regulated at 350 mW. Being a dual headed sorter, the laser beam was split utilizing a CVI Melles Griot high energy 50:50 beam splitter providing 175 mW of power to each head. Power was confirmed at 160 mW at the flow cell using a Power/Energy Meter, model 841-PE. A 70 μm Orient-tip™ was used to generate the sorting streams while using a drop frequency of 68,000 Hz for sorting.

Event rates were held at 30,000 events per second while gating on living and properly oriented sperm, or ˜55-65% of the total cells, while taking ˜40-45% of the X- or Y-region optimized for desired purity by proper region positioning based on the results of the STS analyzer whilst all other live cells excluding all those just mentioned were sent to the third stream. All of this is confirmed using the STS Sexed Semen Purity Analyzer and STS Analyzer Software v.1.0.0., which provides high resolution peaks of X- and Y-chromosome-bearing sperm populations.

The afore-mentioned settings resulted in sorting speeds of 5,000-6,000 sexed sperm per second for X- and Y-chromosome bearing streams. The emphasis on each sort was to achieve either a large percentage (purity) of X- or Y-chromosome-bearing sperm in a given sample with a purity ≧95%. The third stream also resulted in sorting speeds of 5,000-6,000.

The sheath fluid used for these sperm sorting experiments was SortEnsure™. After the sperm have been sorted (sex sorted and conventional sorted) they are collected in 50 ml collection vessels containing catch fluid consisting of a 22% egg yolk-Tris extender. All extenders used in the experiments consisted of the same formulation having a pH of 6.8 and an osmolarity balanced at 300 mOsm for the Tris A extender. Tris B fraction extender is diluted 3:1 (A:B) and vortexed with an osmolarity of 830 mOsm. A deer semen production dose for fresh semen was ˜3×10⁶ sperm per straw (0.25 cc) and ˜6×10⁶ sperm per straw for cryopreserved conventional and sex-sorted straws of semen.

After sorting, all three collected sperm cell samples were collected and cryopreserved in 0.25 cc straws using an automated freezing device, IMV Digitcool and stored under liquid nitrogen. The conventional sample was also cryopreserved using similar storage conditions.

As can be easily understood from the foregoing, the basic concepts of the present invention may be embodied in a variety of ways. As such, the particular embodiments, elements, terms, or expressions disclosed by the description, or shown in the figures, accompanying this application are not intended to be limiting, but rather examples of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “sorter” should be understood to encompass disclosure of the act of “sorting”—whether explicitly discussed or not—and, conversely, were there effectively disclosure of the act of “sorting”, such a disclosure should be understood to encompass disclosure of a “sorter.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

Moreover, for the purposes of the present invention, the term “a” or “an” entity refers to one or more of that entity; for example, “a container” refers to one or more of the containers. As such, the terms “a” or “an”, “one or more” and “at least one” can be used interchangeably herein. Further, as used herein the term “or” means “and/or” unless specifically indicated otherwise.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

The claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

I claim:

1. An apparatus comprising:
a) two or more spaced apart tubes, each tube having;
i) a tube inlet for collecting a stream of droplets in a flow path, wherein the droplets contain particles;
ii) a tube outlet for dispensing the collected droplets;
iii) a tube body connecting the tube inlet to the tube outlet for redirecting the stream of droplets in the flow path;
b) a support holding each tube in a spaced apart relationship; and
c) a securing element for securing the two or more spaced apart tubes to the support.

2. The apparatus as claimed in claim 1, wherein one or more of the spaced apart tubes taper towards the tube outlet.

3. The apparatus as claimed in claim 1, wherein one or more of the spaced apart tubes has at least one bend which is less than 90 degrees.

4. The apparatus as claimed in claim 3, wherein the angle of the bend is in the range of 40 to 80 degrees.

5. The apparatus as claimed in claim 1, further comprising a fastener for locking and unlocking the relative positions of one or more of the spaced apart tubes in the support.

6. The apparatus as claimed in claim 5, further comprising one or more threaded sleeves in the support, and wherein the fastener comprises a screw for engaging one of the threaded sleeves such that a screw tip is able to lock one of the spaced apart tubes into position.

7. The apparatus as claimed in claim 1, wherein one or more of the spaced apart tubes further comprise a tube outlet larger than the tube inlet.

8. The apparatus as claimed in claim 1, wherein the two or more spaced apart tubes comprises at least three spaced apart tubes.

9. The apparatus as claimed in claim 1, further comprising an adjustable X-Y-Z stage connected to the support.

10. The apparatus as claimed in claim 1, wherein one or more of the tube inlets are elevated relative to an upper face of the support.

11. The apparatus as claimed in claim 1, wherein each spaced apart tube is made from an electrically conductive material.

12. The apparatus as claimed in claim 11, wherein the electrically conductive material is selected from the group consisting of: stainless steel, titanium and a biocompatible material.

13. The apparatus as claimed in claim 1, wherein at least a portion of the support comprises an electrically conductive material.

14. The apparatus as claimed in claim 13, wherein the electrically conductive material is selected from the group consisting of: metals, graphite, and electrically conducting polymers.

15. The apparatus as claimed in claim 1, wherein the two or more spaced apart tubes further comprise:
a) a first tube having;
i) a first tube inlet for collecting droplets in a flow path with a first trajectory;
ii) a first tube outlet for depositing droplets from the flow path with the first trajectory to a first collection area;
iii) a first bent tube body connecting the first tube inlet to the first tube outlet for redirecting droplets in the flow path with the first trajectory;
b) a second tube having;
i) a second tube inlet for collecting droplets in a flow path with a second trajectory;
ii) a second tube outlet for depositing droplets from the flow path with the second trajectory to a second collection area;
iii) a second bent tube body connecting the second tube inlet to the second tube outlet for redirecting droplets in the flow path with the second trajectory;
c) a third tube having;
i) a third tube inlet for collecting droplets in a flow path with a third trajectory;
ii) a third tube outlet for depositing droplets from the flow path with the third trajectory to a third collection area; and
iii) a third bent tube body connecting the third tube inlet to the third tube outlet for redirecting droplets in the flow path with the third trajectory.

16. A flow cytometer system to isolate three or more subpopulations of cells, the system comprising:
a) a nozzle for producing a fluid stream along a flow axis;
b) a laser for interrogating cells within the fluid stream;
c) a detector for detecting emitted or reflected electromagnetic radiation from each cell in response to laser interrogation;
d) an analyzer for determining properties of the cells within the fluid stream based on the emitted or reflected electromagnetic radiation, wherein the analyzer identifies cells as either, alive and having a first characteristic, alive and having a second characteristic, alive and undetermined with respect to the first and second characteristics, or dead;
e) an oscillator for perturbing the fluid stream into droplets;
f) a charge circuit for charging droplets as they form;
g) deflection plates for deflecting each droplet according to their charge, wherein the charge is determined by the analyzer to provide droplets containing live cells with the first characteristic with a first trajectory, droplets containing live cells with the second characteristic with a second trajectory, and droplets containing live cells with neither the first nor the second characteristic with a third trajectory;
h) a particle redirection device aligned to received droplets in the first trajectory, droplets in the second trajectory, and droplets in the third trajectory, the particle redirection device comprising:
i) a first tube aligned to receive droplets in the first trajectory and dispense droplets from the first trajectory to a first collection area;
ii) a second tube to receive droplets in the second trajectory and dispense droplets from the second trajectory to a second collection area;
iii) a third tube aligned to receive droplet in the third trajectory and dispense them to a third collection area; and
iv) a support holding each tube in a spaced apart relationship.

17. The system as claimed in claim 16, wherein the system further comprises one or more collection vessels disposed at the tube outlets of each tube and one or more housings adapted to receive the one or more collection vessels, wherein the one or more housings are adapted to be placed in one of two orientations when in use.

18. The system as claimed in claim 16, wherein the system further comprises a video camera for monitoring particle sorting when in use.

19. The system as claimed in claim 16, wherein the number of flow cytometers used at any moment in time is more than the number of collection vessels.

20. A method of sorting sperm cells comprising the steps of:
a) producing a fluid stream containing living sperm cells with a first characteristic, living sperm cells with a second characteristic, and dead sperm cells;
b) detecting properties of sperm cells in the fluid stream;
c) identifying a first subpopulation of sperm cells having the first sperm characteristic;
d) identifying a second subpopulation of sperm cells having the second sperm characteristic;
e) identifying dead sperm cells;
f) identifying a third subpopulation of sperm cells which are neither dead sperm cells, nor can be identified in the first or second subpopulations;
g) collecting the first subpopulation of sperm cells in a first collection vessel;
h) collecting the second subpopulation of sperm cells in a second collection vessel;
i) collecting the third subpopulation of sperm cells in a third collection vessel; and
j) discarding dead sperm cells with the waste.

21. The method as claimed in claim 20, wherein the first sperm characteristic comprises the presences of an X-chromosome, and the second sperm characteristic comprises the presence of a Y-chromosome.

22. The method as claimed in claim 21, wherein the third subpopulation of sperm comprises live sperm which could neither be identified as having an X-chromosome or Y-chromosome.

23. The method as claimed in claim 20, wherein the step of collecting the first subpopulation of sperm further comprises forming a charged droplet and directing the charged droplet along a first trajectory; the step of collecting a second subpopulation of sperm comprises forming a charged droplet and directing the charged droplet along a second trajectory; the step of collecting the third subpopulation of sperm further comprises forming a charged droplet and directing the charged droplet along a third trajectory.

24. The method as claimed in claim 23, further comprising the step of: providing a redirection device for accepting droplets in each of the first trajectory, the second trajectory, and the third trajectory and dispensing the droplets into a respective first collection vessel, second collection vessel and third collection vessel.

25. The method as claimed in claim 20, wherein the sperm cells are selected from a mammal from the group consisting of cervidae, bovidae, elephantidae, equidae, spheniscidae and suidae.

26. The method as claimed in claim 25, wherein the cervidae comprise White-tailed deer, Moose or Eld's deer.
